(19) United States
(12) Patent Application Publication
(10) Pub. No.: US 2014/0365238 A1
(43) Pub. Date: Dec. 11, 2014

(54) BIOLOGICAL INFORMATION DISTRIBUTION SERVER, PROGRAM THEREOF, AND MEDICAL SUPPORT SYSTEM USING THE SAME

(71) Applicant: OMRON HEALTHCARE CO., LTD., Muko-shi (JP)

(72) Inventors: Seiji KONO, Kyoto (JP); Hiroshi OGAWA, Kyoto (JP); Yasuyuki TETSUKA, Kyoto (JP)

(21) Appl. No.: 14/465,270

(22) Filed: Aug. 21, 2014

Related U.S. Application Data
(63) Continuation of application No. PCT/JP2012/080605, filed on Nov. 27, 2012.

(30) Foreign Application Priority Data
Mar. 16, 2012 (JP) 2012-060969

(51) Int. Cl. G06F 19/00; A61B 5/00

(52) U.S. Cl. CPC G06F 19/322; A61B 5/0022
USPC 705/2

(57) ABSTRACT

To provide a medical information distribution server for providing a system for sharing a patient's biological information with a plurality of people having different roles, such as doctors and pharmacists, as well as a medical support system that uses such a server. The medical information distribution server is connected via network to a terminal including a card reader, including: a device for saving an identification symbol assigned to a card and read by the card reader, patient ID for identifying a patient, and biological information of the patient in association with each other; a device for generating display data from information included in the biological information of the patient in accordance with a condition set in advance by a user of the terminal; and a device for transmitting generated display data to a terminal that read the card including the identification symbol, in response to a request from the terminal.

This is a Continuation of International Application No. PCT/JP2012/080605 filed Nov. 27, 2012, which claims the benefit of Japanese Application No. 2012-060969 filed Mar. 16, 2012. The disclosure of the prior applications is hereby incorporated by reference herein in its entirety.

### TECHNICAL FIELD

The present invention relates to biological information distribution servers, programs thereof, and medical support systems using the same.

### BACKGROUND ART

Systems in which biological information such as a blood pressure is saved in a server connected to a network such as the Internet, and the data is then downloaded to and viewed in a terminal as necessary, have been proposed in the past. For example, Patent Literature 1 discloses a “health management support system” in which a “user terminal”, a “health management support system server”, and a “medical staff terminal” are connected to the Internet or the like.

### CITATION LIST

### Patent Literature

Patent Literature 1: JP 2008-225585A

### SUMMARY OF INVENTION

### Technical Problem

A patient's biological information is time-series data measured from day to day for use in situations such as diagnoses, dispensing medications, and so on by specialists such as doctors, and constructing a system in which a patient's measured data is accumulated in a server and distributed to a plurality of user terminals so that specialists such as doctors can easily access that information makes it possible for the biological information to be shared between patients and specialists such as doctors, which is convenient.

However, while the biological information accumulated in the server is time-series information measured from day to day for the patient, the biological information required by a specialist such as a doctor is often not the time-series data itself, but is rather various trends in the data obtained from the measured data as a whole. For example, when a doctor makes a diagnosis for a patient that has been diagnosed in the past, the doctor focuses on data measured during the previous examination, compares that data with trends that have occurred thereafter, and judges the effectiveness of dietary instruction, medications, and so on. Accordingly, it is preferable for the data accumulated in the server to be distributed to respective user terminals having been modified appropriately based on the purpose for which the data will be used.

Having been achieved in light of the aforementioned situation, the present invention relates to a biological information distribution server, program thereof, and medical support system using the same for providing a system for sharing a patient's biological information between the patient and a person aside from the patient such as a doctor.

### Solution to Problem

To achieve the aforementioned object, a medical information distribution server according to the present invention is a medical information distribution server connected via a network to a terminal including a card reader, the medical information distribution server including; a means for saving an identification symbol X uniquely assigned to a card and read by the card reader, a patient ID for identifying a patient, and biological information of the patient in association with each other; a means for generating display data from information included in the biological information of the patient in accordance with a condition set in advance by a user of the terminal; and a means for transmitting the generated display data to a terminal that has read the card including the identification symbol X, in response to a request from the terminal.

Preferably, the medical information distribution server distributes display data that differs for each terminal user by authenticating the user of the terminal in advance prior to receiving the request from the terminal and setting conditions that differ depending on the terminal user.

Preferably, the display data generated by the medical information distribution server includes biological information measured on the day of an examination date and biological information measured on the previous examination date by the patient associated with the identification symbol X. Here, the terminal user is a doctor, for example. Note that this also includes cases where user accounts are assigned to a medical institution as a whole, individual groups therein, and so on.

The display data includes the biological information of the patient and/or information related to a prescription provided by a doctor. Here, the terminal user is a pharmacist, for example. Note that the term “user” also refers to user accounts issued to pharmacies, other establishments, and so on.

The biological information includes a systolic blood pressure of the patient, a diastolic blood pressure of the patient, a heartbeat of the patient, and a measurement date/time on which the measurements were taken. Information such as past illness histories may also be included.

A program according to the present invention causes a computer to function as the aforementioned medical information distribution server.

A medical support system according to the present invention includes: a card in which is recorded a unique identification symbol X assigned on a card-by-card basis; a measurement device for measuring biological information of a patient; a medical information distribution server that receives data of the biological information measured by the measurement device via a network and records the data; and one or more terminals. Here, the measurement device includes a network communication unit having a unique device ID, and the medical information distribution server includes: a means for saving the device ID, the identification symbol X, a patient ID for identifying the patient, and the biological information of the patient in association with each other; a means for generating display data from information included in the biological information of the patient in accordance with a condition set in advance by a user of the terminal; and a means for transmitting the generated display data to a terminal that has read the card including the identification symbol X, in response to a request from the terminal.

### Advantageous Effects of Invention

With the biological information distribution server, program thereof, and medical support system using the same according to the present invention, a system for sharing a patient's biological information between the patient and a person aside from the patient such as a doctor can be provided.

### BRIEF DESCRIPTION OF DRAWINGS

FIG. 1 is a block diagram illustrating the overall configuration of a medical support system according to an embodiment of the present invention.

FIG. 2 is a block diagram illustrating the configuration of a blood pressure meter (a biological information measurement device) according to an embodiment of the present invention.

FIG. 3 is a block diagram illustrating a patient card according to an embodiment of the present invention.

FIG. 4 is a block diagram illustrating a server according to an embodiment of the present invention.

FIG. 5 is a block diagram illustrating a specialist terminal according to an embodiment of the present invention.

FIG. 6 is a block diagram illustrating a patient terminal according to an embodiment of the present invention.

FIG. 7 is a diagram illustrating the configuration of a patient table according to an embodiment of the present invention.

FIG. 8 is a diagram illustrating the configuration of blood pressure data table according to an embodiment of the present invention.

FIG. 9 is a diagram illustrating the configuration of a doctor table according to an embodiment of the present invention.

FIG. 10 is a diagram illustrating the configuration of an examination date table according to an embodiment of the present invention.

FIG. 11 is a diagram illustrating the flow of a blood pressure data storage process according to an embodiment of the present invention.

FIG. 12 is a diagram illustrating the flow of biological information display image data creation according to an embodiment of the present invention.

FIG. 13 is a block diagram illustrating the overall configuration of a medical support system according to another embodiment of the present invention.

### DESCRIPTION OF EMBODIMENTS

### First Embodiment

Hereinafter, an embodiment of the present invention will be described assuming a case where biological information is a blood pressure and a specialist operating a specialist terminal is a doctor.

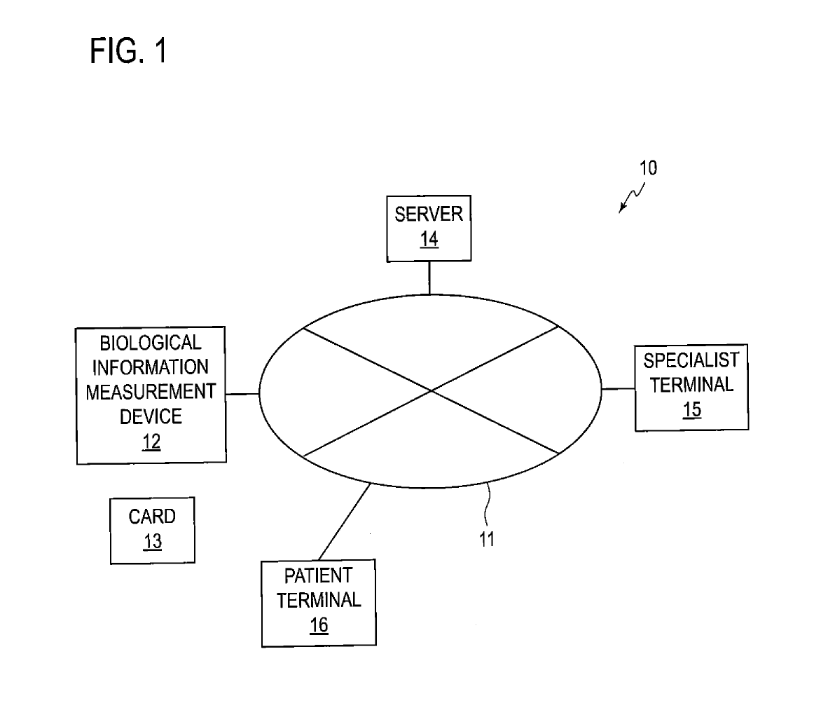

FIG. 1 is a diagram illustrating the overall configuration of a medical support system embodying the present invention. In a medical support system 10 according to the present embodiment, a blood pressure meter (biological information measurement device) 12 capable of measuring and transmitting a blood pressure of a patient, a specialist terminal 15 used by a doctor, a patient terminal 16 used by the patient, and a server 14 are connected over the Internet 11. The server receives the patient's blood pressure from the blood pressure meter and stores the blood pressure in a blood pressure data table in association with the patient and a measurement date/time. The server also receives the examination date from the specialist terminal and stores the examination date in an examination date table in association with the specialist and the patient. Furthermore, each patient carries a card 13 to which is assigned unique card identification information associated with that patient.

The following describes an example of a method for using this medical support system. The patient measures his/her blood pressure each morning and night at home. The measured data is transmitted from the blood pressure meter to the server, and saved and accumulated therein. In the case where the patient visits a hospital and is examined by a doctor periodically (once a month, for example), the doctor can use the specialist terminal to confirm the blood pressure data accumulated in the server and can then examine the patient based on that data.

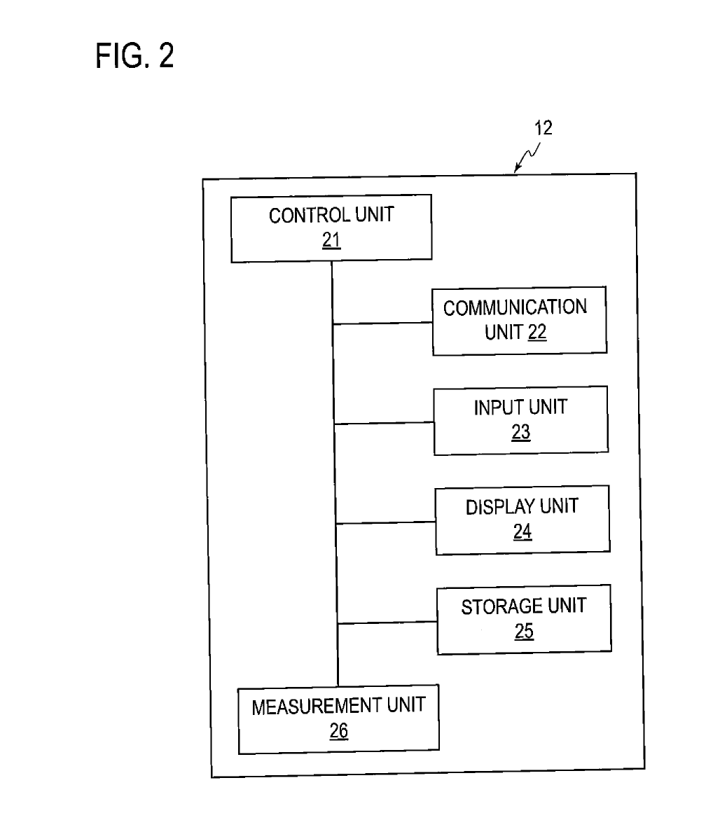

FIG. 2 is a diagram illustrating the configuration of the blood pressure meter 12 according to the present embodiment. The blood pressure meter 12 includes a control unit 21, a communication unit 22, an input unit 23, a display unit 24, a storage unit 25, and a measurement unit 26. The control unit 21 controls the blood pressure meter 12 as a whole. The input unit 23 accepts instructions from the patient, for starting measurement, transmitting data, and so on. The measurement unit 26 measures the patient's blood pressure (systolic blood pressure and diastolic blood pressure) and pulse (this will be collectively called “blood pressure data” hereinafter). The measured blood pressure data is displayed in the display unit 24 and then stored temporarily in the storage unit 25 along with the time of the measurement. When the measurement is complete, the blood pressure data is transmitted to the server 14 via the communication unit 22. The communication unit 22 can use a mobile communication system, for example. In the case where the transmission cannot be completed for some reason, the data continues to be held in the storage unit 25 until the next transmission is carried out and completed.

A unique measurement device identification symbol is electromagnetically recorded in the blood pressure meter. For example, in the case where a 3G mobile telephone function is used as the communication unit, the telephone number thereof can be used as the measurement device identification symbol. The blood pressure meter transmits a set (a record) including the measurement device identification symbol and the measurement date/time and blood pressure data stored in the storage unit to the server.

The measurement device identification symbol may be a device ID unique to the hardware that configures the communication unit of the blood pressure meter 12, such as a Subscriber Identity Module (SIM) card. In this case, if the SIM card is the same, the device ID is also determined to be the same by the server 14, even if the measurement device itself is different.

Meanwhile, in the case where a plurality of patients use the same blood pressure meter, a plurality of buttons may be provided in the blood pressure meter and the individual buttons may be associated with individual patients, for example. When a patient then presses the button corresponding to him/herself when measuring the blood pressure data, the blood pressure meter records the blood pressure data along with a number of the button that has been pressed (also called an “extension symbol” hereinafter). When transmitting the blood pressure data to the server, the blood pressure meter transmits a set including the telephone number and the extension symbol to the server as the measurement device identification symbol. The blood pressure meter can be associated with individual patients by treating the single physical blood pressure meter as a plurality of virtual blood pressure meters, which makes it possible to simplify the overall configuration of the medical support system.

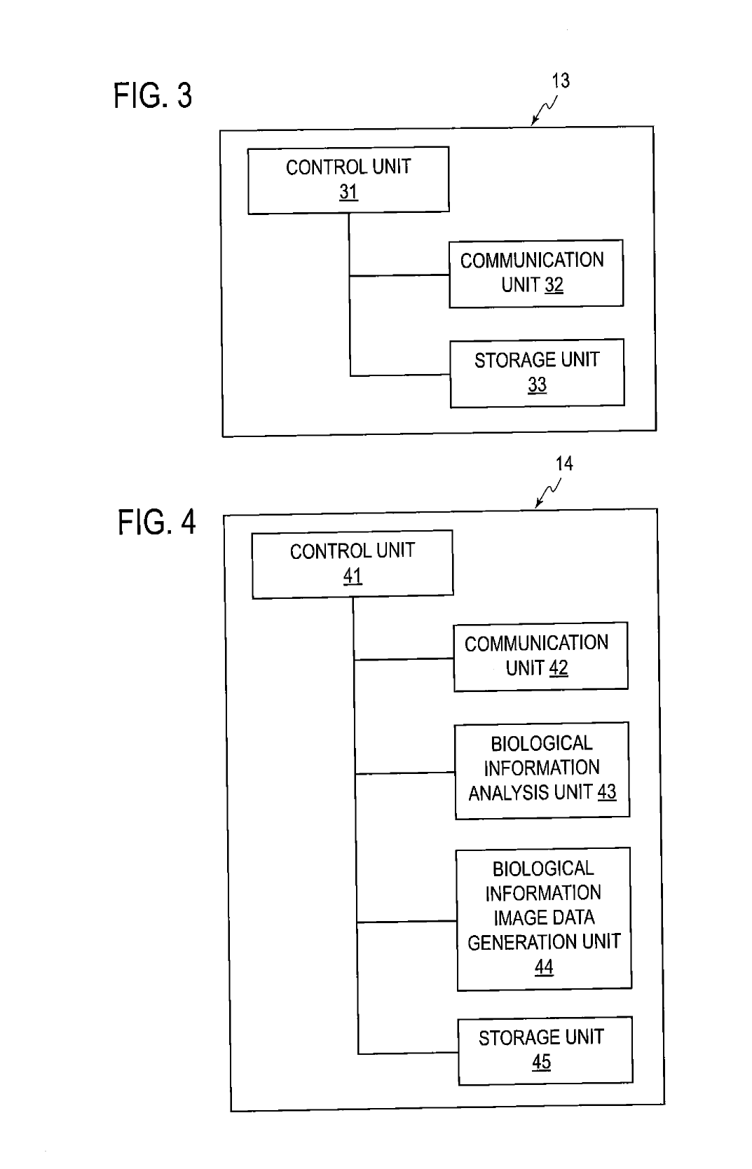

FIG. 3 is a diagram illustrating the configuration of the card 13 carried by the patient. The card 13 includes a control unit 31, a communication unit 32, and a storage unit 33. The control unit 31 controls the card 13 as a whole. A card identification symbol X that is uniquely assigned to the card is electromagnetically recorded in the storage unit 33. The communication unit 32 transmits the card identification symbol X to a card reading unit, mentioned later, that is provided in the specialist terminal 15. A non-contact IC card, for example, can be used as the card 13, and an ID number unique to the IC chip can be used as the card identification symbol X.

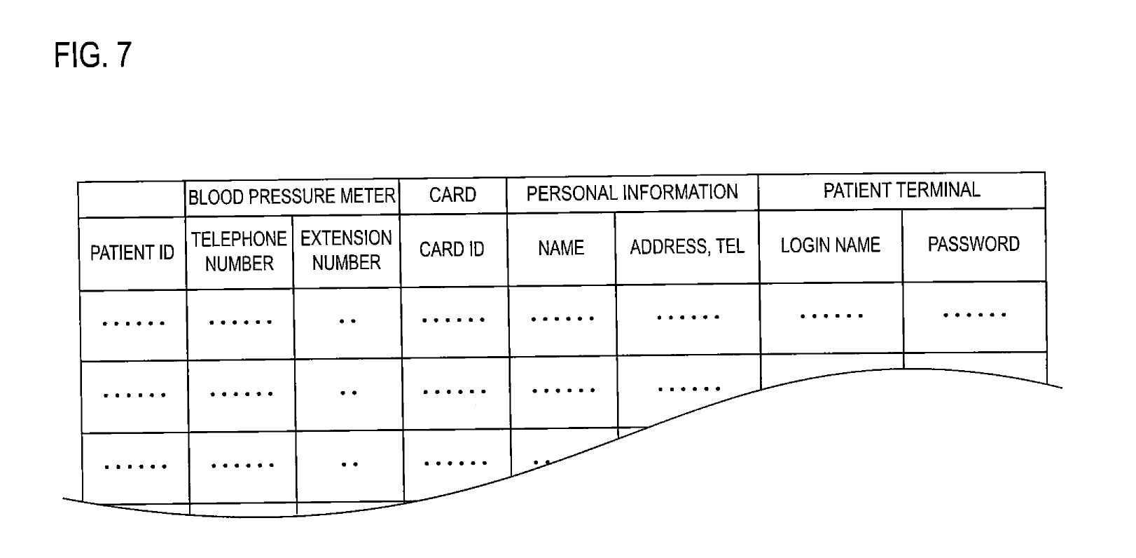

The individual patient, the card identification symbol X of the card 13 carried by the patient, and the measurement device identification symbol of the blood pressure meter 12 (a mobile telephone number and extension symbol) are associated with the blood pressure meter in advance when, for example, the blood pressure meter is sold to the patient, and this information can be stored in the server 14 as a patient table. FIG. 7 illustrates an example of the patient table. The patient table is configured of a plurality of patient records, with each patient record including a patient ID uniquely assigned to each patient; the measurement device identification symbol; the card identification symbol X; personal information such as a name, address, and telephone number; a login name and password used when using the patient terminal; and other information.

FIG. 4 is a diagram illustrating the configuration of the server 14. The server 14 includes a control unit 41, a communication unit 42, a biological information analysis unit 43, a biological information display image data generation unit 44, and a storage unit 45. The control unit 41 controls the server 14 as a whole. The communication unit 42 carries out various types of communication with the blood pressure meter 12, the specialist terminal 15, the patient terminal 16, and so on. The biological information analysis unit 43 performs various types of analyses on the blood pressure data. The biological information display image data generation unit 44 generates image data expressing the blood pressure data and analysis results thereof as a graph. The storage unit 45 stores various types of data, such as the patient table, as well as the blood pressure data, doctor information, and so on.

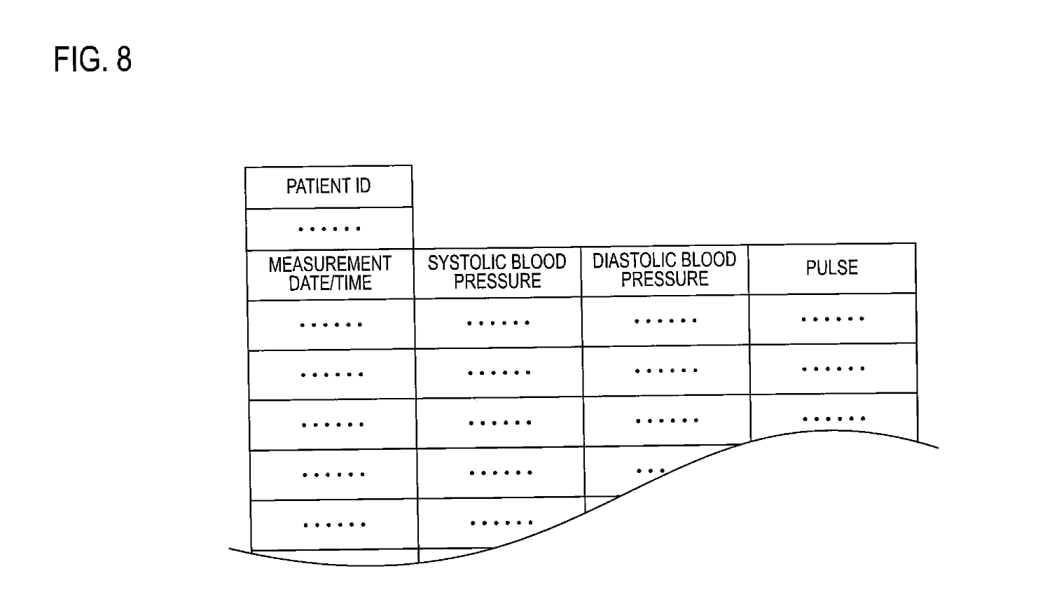

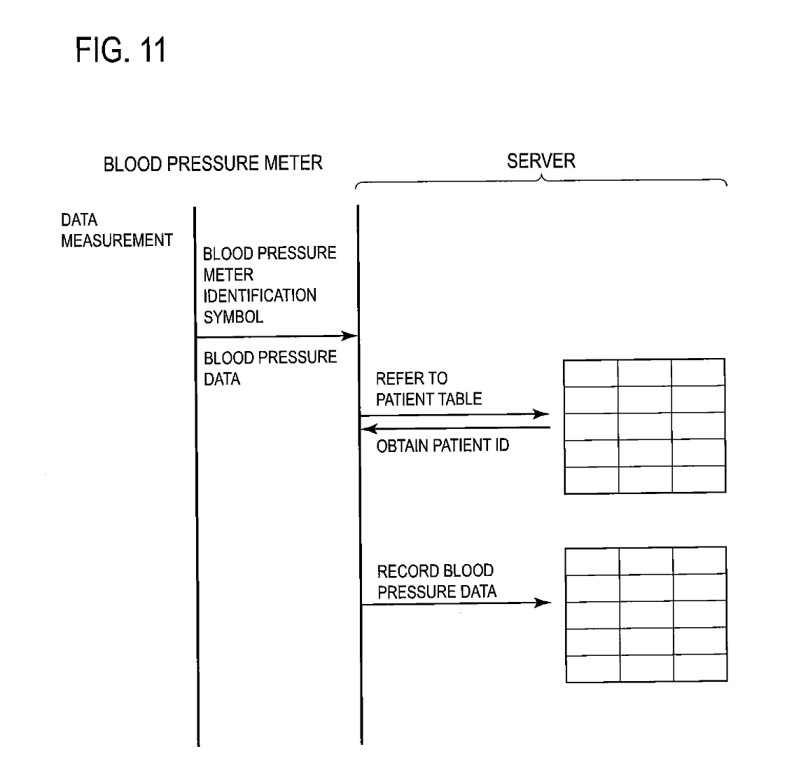

Upon receiving the measurement device identification symbol and the blood pressure data from the blood pressure meter, the server refers to the patient table and obtains the patient ID associated with the measurement device identification symbol. The blood pressure data is also recorded in the blood pressure data table corresponding to the patient ID. FIG. 8 illustrates an example of such a blood pressure data table, and FIG. 11 illustrates the flow of the aforementioned processing. Furthermore, upon receiving a request from the specialist terminal, the patient terminal, or the like, the server confirms the origin thereof, executes processing in accordance with the origin and the details of the request, and transmits the necessary data to the terminal. Details of operations performed by the server will be given later.

The server may be configured from a single computer, or may be configured from a plurality of computers. It is preferable for the server to be configured of a plurality of physical or virtual computers, and for the storage of patient information, biological information, and the like to be carried out by a different computer than a computer that analyzes the biological information and creates the biological information display image data in response to the request from the specialist terminal. This is because doing so makes it possible to flexibly accommodate the replacement/addition of physical units or programs in order to change the details of processes, add functions, and so on.

Note that a case where the server is configured of a plurality of computers will be described in a second embodiment.

The specialist terminal 15 and the patient terminal 16 are provided with functions for displaying display data created by the medical information distribution server 14.

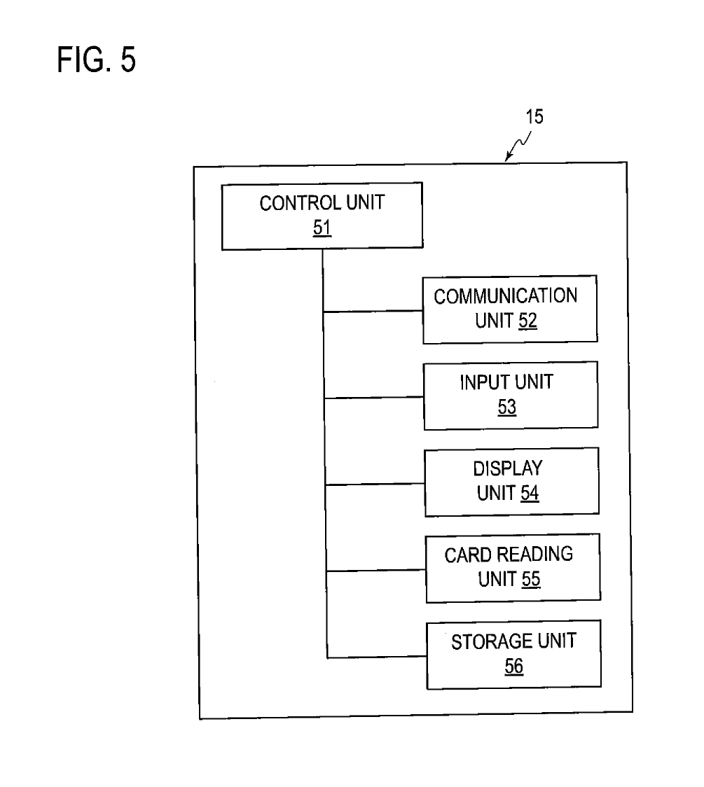

FIG. 5 is a diagram illustrating the configuration of the specialist terminal 15 according to the present embodiment. The specialist terminal 15 includes a control unit 51, a communication unit 52, an input unit 53, a display unit 54, a card reading unit 55, and a storage unit 56. A typical personal computer or the like can be used as the specialist terminal 15. In such a case, units already provided in the typical personal computer can be used as the control unit 51, the communication unit 52, the input unit 53, the display unit 54, and the storage unit 56. The card reading unit 55 of the specialist terminal is a unit for reading the aforementioned card identification symbol X of the card 13 carried by the patient, and can be used by being connected to an external terminal in the personal computer, such as a USB port.

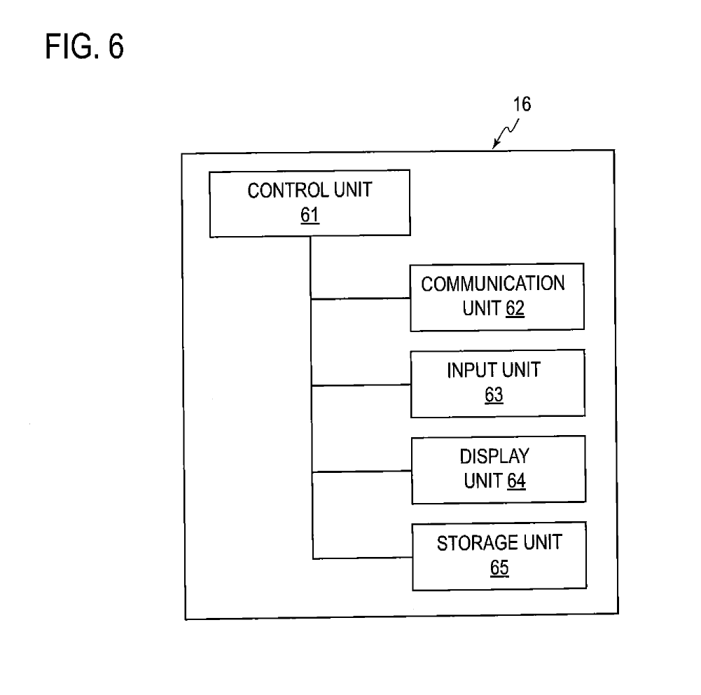

FIG. 6 is a diagram illustrating the configuration of the patient terminal 16 according to the present embodiment. The patient terminal 16 includes a control unit 61, a communication unit 62, an input unit 63, a display unit 64, and a storage unit 65. A typical personal computer or the like can be used as the patient terminal 16. The patient terminal is used for the patient to confirm his/her own past blood pressure data or the like, to change patient information, and so on. The patient can carry out these tasks by using the typical personal computer to log into the server.

Next, a method for displaying the blood pressure data (biological information) in the specialist terminal 15 using the system described thus far will be described in further detail.

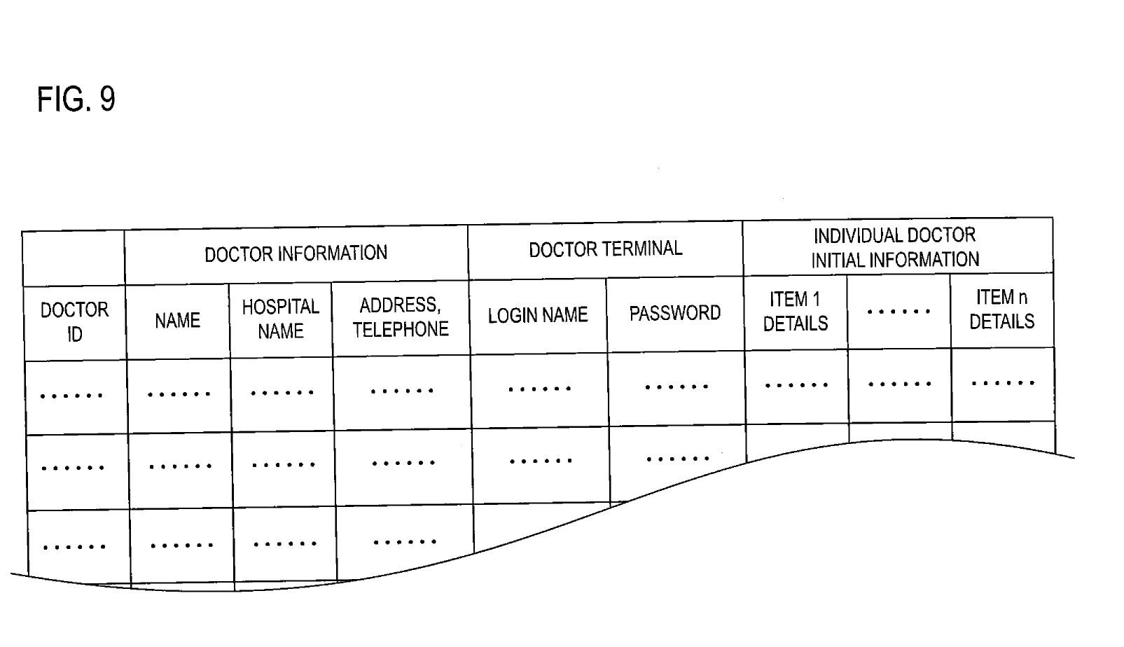

Doctors using the medical support system according to the present embodiment are registered in the doctor table in advance, which is stored in the storage unit 45 of the server 14. FIG. 9 illustrates an example of the doctor table. The doctor table is configured of a plurality of doctor records, with each doctor record including a doctor ID uniquely assigned to each doctor; doctor information such as a name, address, and telephone number; a login name and password used when using the specialist terminal; initial settings regarding examination details; and other information.

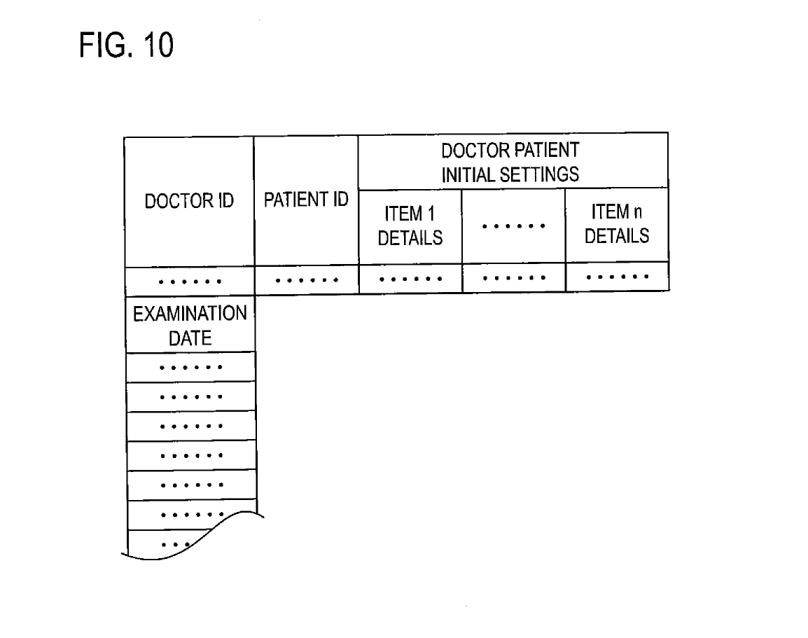

The dates on which a doctor has examined a patient are recorded in the storage unit 45 of the server 14 as an examination date table. FIG. 10 illustrates an example of the examination date table. The examination date table created for each set of a specific doctor and a specific patient, and is configured of the doctor ID; the patient ID; initial settings regarding examination details for each doctor-patient; a list of past examination dates; and other information. Note that the examination date table is created the first time a doctor examines a patient, and thus the server can determine whether that patient has been examined by that doctor in the past based on whether or not the examination date table is present.

The doctor logs into the server 14 from the specialist terminal 15. At this time, a known method can be used for authentication, such as a method that employs a login name and password. The server stores the doctor table in which the doctor IDs uniquely assigned to each of the doctors are associated with the login names and passwords, and refers to the doctor table and obtains the doctor ID when a doctor logs in. When various types of information are then received from the specialist terminal, it can be determined that the information has been transmitted from the doctor having that doctor ID.

In the case where a doctor uses the medical support system for the first time, the doctor is, upon logging into the server 14 from the specialist terminal 15, prompted to make various types of initial settings that are applied to that doctor. A method for selecting blood pressure data, default items in a blood pressure information display screen, a screen design, and so on can be given as examples of such settings. The specialist terminal transmits these settings items and details to the server. Having received these items, the server records the details of the settings in the doctor table stored in the storage unit 45, in association with the doctor ID of the doctor that transmitted the information.

Meanwhile, in the case where the doctor examines a patient carrying the card 13, the above processing can be simplified by causing the card reading unit of the specialist terminal to read the card received from the patient. Upon reading the card using the card reading unit 55, the specialist terminal 15 according to the present embodiment transmits the card identification symbol to the server 14. Here, the card identification symbol functions as the patient's identification information. Upon receiving the card identification symbol, the server refers to the patient table and obtains the patient ID associated with the card identification symbol. The server can further take that date as the examination date and register the examination date in the examination date table corresponding to the doctor ID and patient ID indicating the source of the transmission. Further still, the server can take the received card ID as a biological information transfer request, and start the analysis of the biological information and the creation of the biological information display image data, which will be described later.

Upon receiving the blood pressure data transfer request from the specialist terminal, the server transmits graph display image data to the specialist terminal by carrying out predetermined processing.

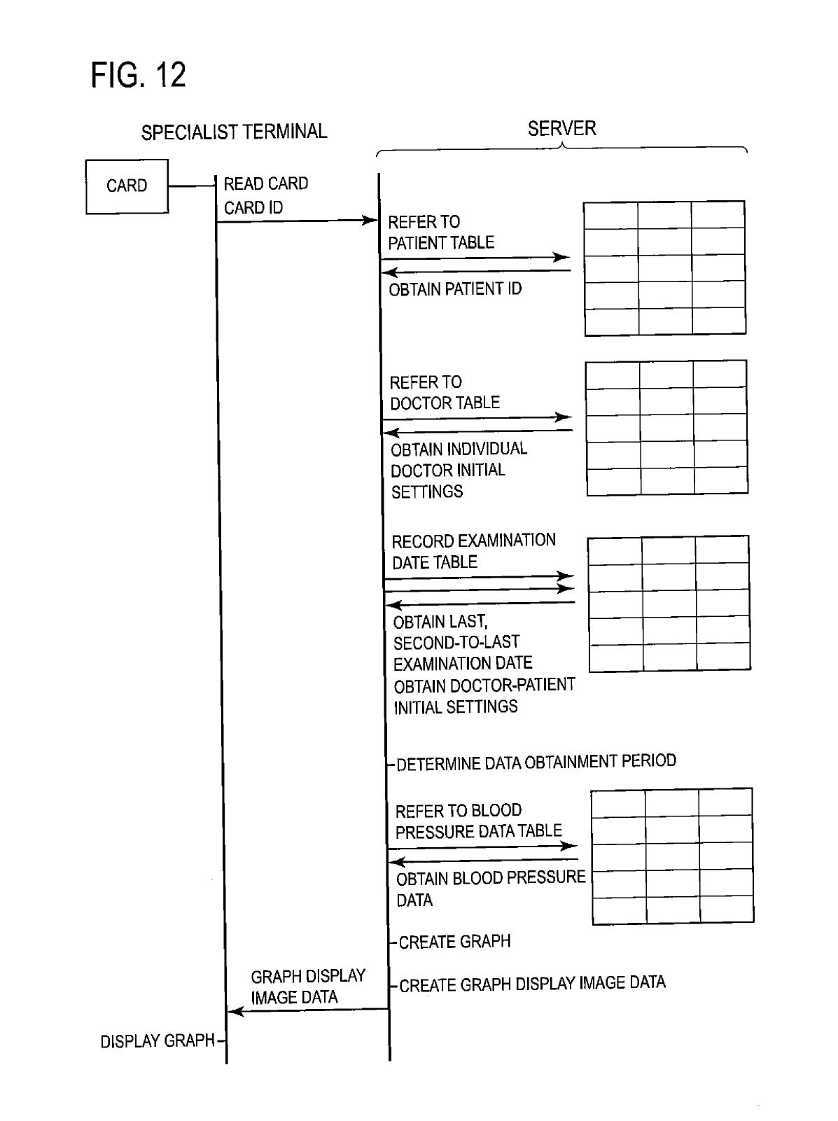

FIG. 12 is a diagram illustrating the flow of biological information display image data creation performed by the server according to the present embodiment. Upon receiving the patient's card ID (the patient's identification information, also serving as a biological information data transfer request) from the specialist terminal, the server carries out the following processing. First, the server refers to the patient table and obtains the patient ID corresponding to the received card ID. Then, the server refers to the doctor table and obtains values of the initial settings for the doctor corresponding to the doctor ID from which the request was transmitted. Next, the server refers to the examination date table corresponding to the doctor ID and the patient ID, and obtains the last examination date and the second-to-last examination date, along with the doctor-patient initial setting values. Then, based on the obtained second-to-last examination date, the server determines a period of the blood pressure data to display (also called a “display period” hereinafter). This display period includes a period spanning at least from the second-to-last examination date to the date on which the biological information data transfer request was received (called a “current examination date” hereinafter). Next, the server obtains the blood pressure data for that display period from the blood pressure data table corresponding to the patient ID. Then, the server creates a graph expressing the obtained blood pressure data in time series (a transitive graph) and creates display image data along therewith. At this time, the display image data is created so that the second-to-last examination date and the last examination date are also displayed in the graph. The display image data may be created so that a comparison reference value is also displayed in the graph. When the image data of the transitive graph has been created in this manner, the server transmits the image data to the specialist terminal. Using this graph, the doctor can confirm transitions in the biological information data from the second-to-last examination date, and can more precisely understand a relationship between the diagnoses, procedures, treatments, prescriptions, and so on made on each past examination date.

The data accumulated in the server in this manner is then distributed in response to a request from the specialist terminal. A specialist such as a doctor can cause the data in the server to be displayed in a display screen of the specialist terminal by causing the card carried by the patient to be read by the card reading unit of the specialist terminal, and the patient's biological information can be shared between the patient and a person aside from the patient, such as the doctor. Furthermore, in addition to simply saving and accumulating the patient's biological information in time series, the server can carry out predetermined processing on the patient's biological information in order to distribute the information having been modified appropriately based on the purpose for which the data will be used.

The present invention is not intended to be limited to the embodiment described thus far. Although the embodiment describes the biological information as a blood pressure value and the specialist as a doctor, the present invention is not limited thereto. In addition to a blood pressure, the biological information may be any type of biological information measured from day to day by a patient and required by a doctor for a diagnosis, such as a blood sugar level or the like. Furthermore, the specialist may be a medical worker aside from a doctor, or another type of specialist.

### Second Embodiment

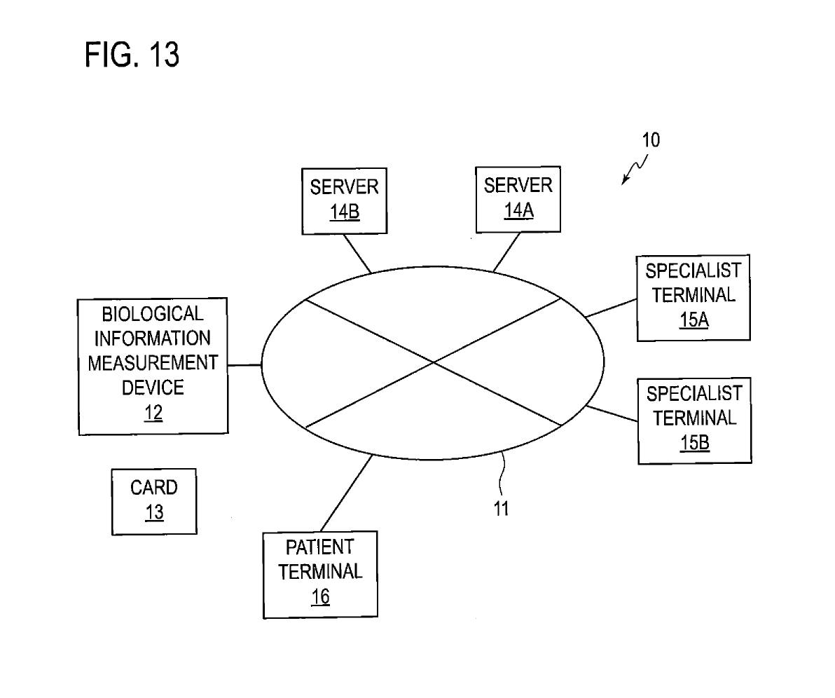

FIG. 13 is a diagram illustrating the overall configuration of a medical support system embodying the present invention. The basic configuration is the same as described in the first embodiment, and identical configurations have been assigned identical reference numerals.

In the medical support system 10 according to the present embodiment, a blood pressure meter (biological information measurement device) 12 capable of measuring and transmitting a blood pressure of a patient, a plurality of specialist terminals 15 (15A and 15B), a patient terminal 16 used by the patient, and servers 14 (also called a first server 14A and a second server 14B) are connected over the Internet 11.

The servers 14 are configured of a plurality of physically or virtually separate computers. The first server 14A handles the analysis of the biological information and the creation of the biological information display image data in response to a request from a specialist terminal. The second server 14B, on the other hand, primarily handles the storage of patient information, biological information, and so on.

The specialist terminals 15 request the first server 14A to transmit necessary data. In response to this request, the first server 14A analyzes the biological information and generates the biological information display image data, and distributes the data to the specialist terminals 15. In the case where there are a plurality of specialist terminals, it is preferable for the appropriate information to be distributed in accordance with the users of the specialist terminals. This is because in the case where, for example, the same patient is examined by different doctors, the courses of treatment, basis for diagnoses, and so on can differ from doctor to doctor.

A case where the specialist is a pharmacist (or pharmacy) rather than a doctor can also be considered. In such a case, the “doctor ID” described in the first embodiment can be replaced with a “specialist ID”, and the pharmacist can be included as a user of the specialist terminal. That is, the server stores a specialist table in which the specialist IDs, which are uniquely assigned to respective specialists, are associated with login names and passwords, and the server refers to the specialist table when a specialist logs in and obtains the specialist ID, thus executing authentication when the specialist terminal 15 logs into the server 14. Furthermore, when various types of information are then received from the specialist terminal, it can be determined that the information has been transmitted from the specialist having that specialist ID.

A pharmacist logs into the medical information distribution server 14B through the terminal 15B in advance, which is set to be used by a pharmacist. When a patient brings his/her prescription and card, the pharmacist can access the patient's biological information by using the card reading unit 55 to read the card 13 received from the patient. Doing so enables the pharmacist to more specifically understand the patient's illness to a necessary extent, and is also useful in preventing human error such as providing a different medication than that specified in the prescription. It also becomes possible for the pharmacy to provide a service in which the patient's measured biological information data is printed, collected in a handbook, or the like. Thus far, from the patient's perspective, any pharmacy has been acceptable as long as the patient's prescription can be filled. However, a pharmacy that has a terminal including the card reading unit 55 can distinguish itself from other pharmacies by providing a “blood pressure handbook printing service”, which from the patient's perspective is a service that adds value.

Furthermore, a patient typically visits a pharmacy with a prescription after being examined by a doctor, and thus if the doctor saves information regarding the prescription in the server 14, the information regarding the prescription can then be shared between the pharmacist and the patient. Doing so makes it possible to eliminate a step of printing out the prescription, which contributes to a reduction in the use of paper. This system has a further benefit in that it enables the correct prescription to be confirmed through double-checking, even in the case where the prescription held by the patient is incorrect due to compounded human error.

The patient terminal 16 requests permission to browse records of biological information saved and accumulated in the second server. In response to this request, the second server distributes the time-series biological information data to the patient terminal 16.

As described above, the second server 14B saves the unique identification symbol X assigned to each card, the patient ID for identifying the patient, and the biological information of the patient in association with each other. On the other hand, by obtaining some of the necessary information from the second server and carrying out necessary processes, the first server 14A generates display data in accordance with conditions set in advance by the users of the terminals, and transmits, to the terminals, display data generated in response to the request from the terminal that has read the card having the identification symbol X. Providing such a system makes it possible to realize a medical support system that enables a patient's biological information to be shared among the patient, a doctor, and a pharmacist.

1. A medical information distribution server connected via a network to a terminal including a card reader, the medical information distribution server comprising:
a means for saving an identification symbol X uniquely assigned to a card and read by the card reader, a patient ID for identifying a patient, and biological information of the patient in association with each other;
a means for recording setting information associated with a unique user ID assigned to each user of the terminal and generating display data from information included in the biological information of the patient in accordance with the setting information that has been set by the user of the terminal in advance; and
a means for transmitting the generated display data to a terminal that has read the card including the identification symbol X, in response to a request from the terminal.

2. A medical information distribution server connected via a network to a terminal including a card reader and to a biological information sharing server that saves an identification symbol X uniquely assigned to a card and read by the card reader, a patient ID for identifying a patient, and biological information of the patient in association with each other, the medical information distribution server comprising:
a means for recording setting information associated with a unique user ID assigned to each user of the terminal and generating display data in accordance with the setting information set in advance by a user of the terminal by obtaining some of information included in the biological information of the patient from the biological information sharing server; and
a means for transmitting the generated display data to a terminal that has read the card including the identification symbol X, in response to a request from the terminal.

3. The medical information distribution server according to claim 1,
wherein the medical information distribution server distributes display data that differs for each terminal user by authenticating the user of the terminal in advance prior to receiving the request from the terminal and setting conditions that differ depending on the terminal user.

4. The medical information distribution server according to claim 1,
wherein the display data generated by the medical information distribution server includes biological information measured on the day of an examination date and biological information measured on the previous examination date by the patient associated with the identification symbol X.

5. The medical information distribution server according to claim 4,
wherein the display data includes the biological information of the patient and/or information related to a prescription provided by a doctor.

6. The medical information distribution server according to claim 1,
wherein the biological information includes a systolic blood pressure of the patient, a diastolic blood pressure of the patient, and a measurement date/time on which the blood pressures were measured.

7. A program for causing a computer to function as the medical information distribution server according to claim 1.

8. A medical support system comprising:
a card in which is recorded a unique identification symbol X assigned on a card-by-card basis;
a measurement device for measuring biological information of a patient;
a medical information distribution server that receives data of the biological information measured by the measurement device via a network and records the data; and
one or more terminals,
wherein the measurement device includes a network communication unit having a unique device ID; and
the medical information distribution server includes:
a means for saving the device ID, the identification symbol X, a patient ID for identifying the patient, and the biological information of the patient in association with each other;
a means for recording setting information associated with a unique user ID assigned to each user of the terminal and generating display data from information included in the biological information of the patient in accordance with the setting information that has been set by the user of the terminal in advance; and
a means for transmitting the generated display data to a terminal that has read the card including the identification symbol X, in response to a request from the terminal.
